(19) United States
(12) Patent Application Publication
(10) Pub. No.: US 2021/0346380 A1
(43) Pub. Date: Nov. 11, 2021

(54) TREATMENT OF SKIN DISORDERS WITH COMPOSITIONS COMPRISING AN EGFR INHIBITOR

(71) Applicant: Sol-Gel Technologies Ltd., Ness Ziona (IL)

(72) Inventors: Moshe ARKIN, Kfar Shmaryahu (IL); Marcel ZIGHELBOIM, Kiryat Motzkin (IL); Ori NOV, Tarum (IL); Ofer TOLEDANO, Kfar Saba (IL); Karine NEIMANN, Ness Ziona (IL); Asher Cachlon, Ashkelon (IL)

(73) Assignee: Sol-Gel Technologies Ltd., Ness Ziona (IL)

(21) Appl. No.: 17/352,322

(22) Filed: Jun. 20, 2021

Related U.S. Application Data
(63) Continuation-in-part of application No. 16/737,503, filed on Jan. 8, 2020.
Application No. 16/737,503 is a continuation-in-part of application No. PCT/IL2019/051410, filed on Dec. 25, 2019.
(60) Provisional application No. 62/877,957, filed on Jul. 24, 2019.
(60) Provisional application No. 62/877,990, filed on Jul. 24, 2019.
(60) Provisional application No. 62/784,738, filed on Dec. 25, 2018.

(51) Int. Cl. A61K 31/517; A61K 9/00; A61K 45/06

(52) U.S. Cl. CPC A61K 31/517; A61K 45/06; A61K 9/0019; A61K 9/0014

(57) ABSTRACT

This invention relates to compositions and methods of treatment of skin or mucosal disorders by administration of compositions comprising at least one EGFR inhibitor, such as topical compositions comprising erlotinib. The compositions of this invention are useful for the treatment, prevention or amelioration of skin or mucosal disorders like psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa.

### CROSS REFERENCE TO RELATED APPLICATIONS

This application is a Continuation-in-Part Application of U.S. application Ser. No. 16/737,503, filed Jan. 8, 2020, which is a Continuation-in-Part of PCT International Patent Application No. PCT/IL2019/051410, filed on Dec. 25, 2019, which claims the benefit of U.S. Provisional Application Ser. No. 62/877,957, filed on Jul. 24, 2019, U.S. Provisional Application Ser. No. 62/877,990, filed on Jul. 24, 2019, and U.S. Provisional Application Ser. No. 62/784,738, filed Dec. 25, 2018, which are all incorporated in their entirety herein by reference.

### FIELD OF THE INVENTION

This invention, in some embodiments thereof, relates to compositions and methods of treatment of skin or mucosal disorders by administration of compositions comprising at least one EGFR inhibitor, such as topical compositions comprising erlotinib. The compositions of this invention are useful for the treatment, prevention or amelioration of skin or mucosal disorders like psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa.

### BACKGROUND

Epidermal Growth Factor Receptor (EGFR) inhibitor drugs like erlotinib, gefitinib, osimertinib and brigatinib target the EGFR (a known oncogene) and are used for the systemic treatment of some forms of cancer (lung, colon).

There is no US-marketed EGFR inhibitor drug for topical use or for injection. The EGFR inhibitor erlotinib is sold as oral tablets (Tarceva). Similarly, gefitinib (Iressa), osimertinib (Tigresso) and brigatinib (Alunbrig) are sold as oral tablets.

There is an unmet need for methods of topical or injectable treatment of skin or mucosal disorders like psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa.

### SUMMARY OF THE INVENTION

In one embodiment, this invention is directed to a topical composition for treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa comprising at least one EGFR inhibitor selected from the group consisting of erlotinib, gefitinib, lapatinib, cetuximab, panitumumab, vandetanib, necitumumab, osimertinib or a pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w.

In one further embodiment, this invention is directed to a topical composition for treatment, prevention or alleviation of a hyperkeratinization disorder in a patient in need thereof, comprising about 0.75% w/w erlotinib hydrochloride and from about 10% to about 98% w/w at least one penetration enhancer, or at least one keratolytic agent, or combination thereof, wherein the composition is formulated as a gel. In another embodiment, the composition comprises between 0.1%-40% w/w keratolytic agent.

In one further embodiment, this invention is directed to a topical composition or by injectable administration for treatment, prevention or alleviation of psoriasis in a patient in need thereof, comprising from about 0.5% to about 20 wt % erlotinib hydrochloride and from about 10% to about 98% w/w at least one penetration enhancer, or at least one keratolytic agent, or combination thereof, wherein the composition is formulated as an ointment. In another embodiment, the composition comprises between 0.1%-40% w/w keratolytic agent.

In one further embodiment, this invention is directed to a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In one further embodiment, this invention is directed to a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tofacitinib citrate and from about 10% w/w to about 98% w/w at least one penetration enhancer, or at least one keratolytic agent, or combination thereof. In another embodiment, the composition comprises between 0.1%-40% w/w keratolytic agent.

In one further embodiment, this invention is directed to a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% at least one penetration enhancer, or at least one keratolytic agent, or combination thereof. In another embodiment, the composition comprises between 0.1%-40% w/w keratolytic agent.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w.

In some embodiments, this invention provides a method of treatment, prevention or alleviation of psoriasis, by topical or injectable administration to a subject in need thereof erlotinib or pharmaceutically acceptable salt thereof in a concentration of from about 0.5% to about 20% w/w.

In other embodiments, this invention provides a method of treatment, prevention or alleviation of psoriasis, by topical or injectable administration to a subject in need thereof erlotinib or pharmaceutically acceptable salt thereof in a concentration of from about 0.5% to about 20% w/w, wherein said method does not induce cutaneous toxicity, or induces reduced cutaneous toxicity of erlotinib or pharmaceutically acceptable salt thereof.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
(i) a composition comprising a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, wherein the two separate compositions are administered concomitantly or sequentially, in either order.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof, in a concentration of 0.5% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of

a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, 0.01% to about 1%, from about 1% to about 3%, from about 3% to about 5% w/w, from 4% to about 6% w/w, from about 5% to about 10% w/w or from about 10% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In another embodiment, the method further comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
(i) a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w,
wherein the two separate compositions are administered concomitantly or sequentially, in either order.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof, in a concentration of from 0.5% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof

a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w.

In another embodiment, the method further comprises administering a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof
(i) a composition comprising a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable slat thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w;
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof in a concentration of from about 0.01% to about 1%, from about 1% to about 3%, from about 3% to about 5% w/w;
wherein the three separate compositions are administered concomitantly or sequentially, in either order.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof in a concentration of from about 0.5% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of

a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 10%, from about 1% to about 20%, from about 0.01% to about 1%, from about 1% to about 3%, from about 3% to about 5% w/w, from 4% to about 6% w/w, from about 5% to about 10% w/w or from about 10% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

### DETAILED DESCRIPTION OF THE INVENTION

Treatment with EGFR inhibitors in general and erlotinib in particular is known to induce cutaneous conditions like acneiform rash, papulopustular rash, abnormal scalp hair growth, abnormal facial hair growth, abnormal hair growth, abnormal eyelash growth, paronychia with or without pyogenic granulomas and telangiectasia.

This is probably one of the reasons that no topical EGFR inhibitor product is developed or marketed so far. A number of clinical studies are underway on the topical treatment or prevention of the EGFR inhibitors-induced cutaneous side-effects, but none on treatment of skin disorders by administration of topical EGFR inhibitors.

It occurred to the present inventors that EGFR inhibitors, being tyrosine kinase inhibitors and also essential regulators of multiple epidermal functions have the potential to prevent, cure or alleviate a number of skin or mucosal disorders in which tyrosine kinase inhibition or epidermal function regulation play a causal mechanistic role. In addition, there are advantages in topically treating skin disorders by topical or injectable instead of systemic administration, thus avoiding systemic side-effects, provided the cutaneous EGFR inhibitors side-effects can be avoided, prevented or minimized.

Surprisingly, according to the present invention, even by increasing the EGFR inhibitors (such as erlotinib) concentration up to 5 wt % or even up to 10 wt %, the cutaneous side effects were not increased.

The EGFR inhibitor cutaneous side-effects reported in the medical literature are the result of oral (systemic) treatment with EGFR inhibitors. The compositions and methods of treatment of this invention use non-oral administration, thus avoiding systemic effects, and are therefore expected to present an advantageous cutaneous side-effects profile as compared to the EGFR inhibitor oral products.

Some of the skin or mucosal disorders contemplated for treatment with the methods of this invention are psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa (see below).

Psoriasis

Psoriasis is characterized i.a. by epidermal hyperproliferation. Protein tyrosine kinases (PTKs) regulate cell proliferation, differentiation and immune processes. Uncontrolled signaling from receptor and intracellular tyrosine kinases can lead to numerous proliferative diseases, i.a. psoriasis (Ben-Bassat H et al Curr. Pharm Des. 2000 June; 6(9):933-42).

### Palmoplantar Psoriasis

Palmoplantar psoriasis is a variant of psoriasis that characteristically affects the skin of the palms and soles. It features hyperkeratotic, pustular, or mixed morphologies. The condition is chronic in nature and produces significant functional disability (see Miceli A, Schmieder G J. Palmoplantar Psoriasis. [Updated 2019 Jun. 3]. StatPearls [Internet]. Treasure Island (FL): StatPearls Publishing; 2019 January). https://www.ncbi.nlm.nih.gov/books/NBK448142/

### Non-Melanoma Skin Cancer (NMSC)

Skin cancers include three main types—basal-cell skin cancer (BCC), squamous cell skin cancer (SCC) and melanoma.

The first two types together (BCC and SCC) are known as non-melanoma skin cancers (NMSC).

Cetuximab (Erbitux), an EGFR inhibitor, has been investigated for oral treatment of NMSC (Wollina U., Expert Opinion on Biological Therapy, Vol. 14, 2014—Issue 2).

Uncontrolled signaling from receptor and intracellular tyrosine kinases can lead to numerous proliferative diseases, i.e. cancer (Ben-Bassat H et al Curr. Pharm Des. 2000 June; 6(9): 933-42).

### Actinic Keratosis (AK)

Dysregulation of the EGFR signaling results in cellular hyperproliferation and defects in differentiation (Joseph S R et al., “Dysregulation of epidermal growth factor receptor in actinic keratosis and squamous cell carcinoma”, Curr Probl. Dermatol. 2015; 46:20-7).

### Gorlin Syndrome (NBCCS)

NBCCS (Nevoid Basal-Cell Carcinoma Syndrome) is i.e. a predisposition for BCC caused by a genetic mutation. Oral treatment of NMSC (which includes BCC) with cetuximab (an EGFR inhibitor) has been investigated, but the topical treatment of Gorlin syndrome with topical EGFR inhibitors was never attempted.

### Hidradenitis Suppurativa (HS)

Hidradenitis suppurativa (HS), also known as acne inversa, is a long-term chronic skin disease whose present treatment options are often unsatisfactory. HS has a profound effect on patient's quality of life (QoL). Alavi A. et al., reviewed QoL aspects of this disease in an article titled “Quality-of-Life Impairment in Patients with Hidradenitis Suppurativa” (Am J Clin Dermatol, 2014, vol. 15. No. 6).

The clinical picture of HS includes solitary nodules, diffuse, painful abscesses, malodorous drainage, sinus tract formation and scarring.

The exact cause of hidradenitis suppurativa is still unclear, but it is believed that the underlying mechanism involves dysfunction of the apocrine sweat glands or hair follicles.

### Pachyonychia Congenita

Pachyonychia Congenita (PC) is an ultra-rare genetic autosomal dominant skin disorder. PC is caused by a mutation in one of five keratin genes KRT6A, KRT6B, KRT6C, KRT16 or KRT17. Keratin genes are responsible for production of keratins, which are tough, fibrous proteins that form filaments to support skin cells and give them shape and strength. Keratin filaments help cells handle pressure and stretching. With PC, the filaments do not form properly, causing extreme cell fragility. PC is a congenital autosomal dominant syndrome primarily affecting males, mainly characterized by increased thickness of the nails, hyperkeratosis involving the palms, soles, knees, and elbows, with popular tiny cutaneous horns, leukoplakia of the mucous membranes (leukokeratosis of the oral mucosa), and usually excessive sweating of the hands and feet; associated with development of bullae on palms and soles after trauma, also characterized by cysts of various types (including steatocystoma and pilosebaceous cysts), characterized by follicular hyperkeratosis (FHK or bumps around hairs at friction sites such as waist, hips, knees, elbows). Most common in children and lessens after teenage years.

In some embodiments, the PC is associated with nail dystrophy. In other embodiments the PC is associated with a keratinization skin disorder.

### Keratinization Skin Disorders

This class of skin disorders includes hyperkeratinization disorder, Darier's disease, Hailey-Hailey disease, erythrodermic autosomal recessive lamellar ichthyosis, nonerythrodermic autosomal recessive lamellar ichthyosis, autosomal dominant lamellar ichthyosis, bullous congenital ichthyosiform erythroderma, palmoplantar keratoderma, erythrokeratodermia variabilis, verrucous epidermal nevi, pityriasis rubra pilaris, Netherton syndrome, idiopathic vulgaris, ichthyosis vulgaris, monilethrix, keratosis piliaris, bullous ichthyosiform erythroderma, nonbullous congenital ichthyosis, Sjogren-Larsson syndrome, erythrokeratodermica variabilis, hyperkeratosis lenticularis perstans, eythrokeratodermia figurate variabilis, mutilating keratoderma of Vohwinkel, Harlequin ichthyosis and Tay's syndrome.

A new terminology for the keratinization skin disorders has been recently introduced (see Akiyama M. et al., J Dermatol Sci. 2018 May; 90(2):105-111, “Autoinflammatory keratinization diseases: An emerging concept encompassing various inflammatory keratinization disorders of the skin”).

In one embodiment the keratinization skin disorder includes Pachyonychia Congenita (PC).

### Keratinization Mucosal Disorders

This class of mucosal (oral, vaginal, anal) disorders includes Lichen Planus, Leukoplakia and Lichen sclerosus.

### Prurigo Nodularis

Prurigo nodularis is a skin disease characterised by pruritic (itchy) nodules which usually appear on the arms or legs. Patients often present with multiple excoriated lesions caused by scratching. Prurigo nodularis is very hard to treat, but current therapies include steroids, vitamins, cryosurgery, thalidomide and UVB light.

### Prurigo Pigmentosa

Prurigo pigmentosa is a rare skin condition of unknown cause, characterized by the sudden onset of erythematous papules that leave a reticulated hyperpigmentation when they heal.

In some embodiments, the EGFR inhibitor in this invention is selected from gefitinib, erlotinib, lapatinib, cetuximab, panitumumab, vandetanib, necitumumab, osimertinib and combinations thereof.

According to some embodiments, there are provided novel methods of treatment of above enumerated skin or mucosal disorders by topical administration of at least one EGFR inhibitor.

In some embodiments there is provided a method of treatment of a skin or mucosal disorder in which epidermal function regulation or tyrosine kinase inhibition play a causal mechanistic role, by topical administration of a therapeutically effective amount of at least one EGFR inhibitor.

According to some embodiments, there is provided a topical composition for treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa, comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w. In another embodiment, the keratinization skin disorder is hyperkeratinization disorder. Each possibility represents a separate embodiment of this invention. In some embodiments, the topical composition further comprises at least one penetration enhancer. In one embodiment, the penetration enhancer is in a concentration of between 10% w/w to about 98% w/w of said composition. In some embodiments, the topical composition further comprises at least one keratolytic agent. In one embodiment, the keratolytic agent is in a concentration of between 0.1% w/w to about 40% w/w of said composition.

In one other embodiment, the at least one penetration enhancer is selected from: DMSO (dimethyl sulfoxide), ethanol, isopropyl alcohol, dimethyl isosorbide, isopropyl myristate, oleic acid, a polyethylene glycol, hexylene glycol, glycofurol and combinations thereof. In one another embodiment, the at least one penetration enhancer has dual functionality and may act also as solvent. Each possibility represents a separate embodiment of this invention.

In some embodiments, the composition of this invention and method of use thereof comprises a keratolytic agent.

As used herein a “keratolytic agent” refers to a compound which loosens and removes the stratum corneum of the skin or alters the structure of the keratin layers of skin. Suitable keratolytic agents include alpha-hydroxy acids. Non-limiting examples of alpha-hydroxy adds include lactic acid and glycolic acid, malic acid, citric acid and tartaric acid. Alfa hydroxyl acids are keratolytic, and they are also capable of trapping moisture in the skin and initiating the formation of collagen. Another group of keratolytic agents, suitable for inclusion in the therapeutic composition according to the present invention is beta-hydroxy acids, such as Salicylic acid (o-hydroxybenzoic acid). Beta hydroxyl acids are keratolytic, and they are also have anti-inflammatory and antibacterial properties. Short chain carboxylic acids (carboxylic acids having up to 6 carbon atoms in their skeleton) are also suitable for inclusion in the therapeutic composition as keratolytic agents. Examples of short chain carboxylic acid include, but are not limited to formic acid, acetic acid, propionic acid, butyric acid (Butanoic acid), valeric acid (pentanoic acid) and caproic acid (hexanoic acid). Also suitable under the definition of short chain carboxylic acid are unsaturated short chain carboxylic acids, i.e., short chain carboxylic acids, having one or more double bonds in their carbon skeleton; and halogenated short chain carboxylic acids, such as fluoroethanoic acid (CH₂FCO₂H), chloroethanoic acid (CH₂ClCO₂H) and dichloroethanoic acid (CHCl₂CO₂H). In preferred embodiments, the short chain carboxylic acid is selected from the list consisting of formic acid, acetic acid, propionic acid, butyric acid. Another group of keratolytic agents include phenol and substituted phenolic compounds. Such compounds are known to dissolve and loosen the intracellular matrix of the hyperkeratinized tissue. Dihydroxy benzene and derivatives thereof have been recognized as potent keratolytic agents. Resorcinol (m-dihydroxybenzene) and derivatives thereof are used in anti-acne preparations. Hydroquinone (p-dihydroxybenzene), besides its anti-pigmentation properties, is also keratolytic. Yet, another class of preferred keratolytic agents includes urea and derivatives thereof. Urea possesses both keratolytic and skin-hydration properties which are beneficial to the damaged tissue of the skin.

In other embodiment, the keratolytic agent used within the composition of this invention and method of use thereof is selected from the group consisting of benzoyl peroxide, coal tar, dithranol, salicylic acid, selenium disulfide, alpha-hydroxy acid, urea, lactic acid, boric acid, retinoic acid, sodium thioglycolate, allantoin, zinc pyrithione, zinc L-pyrrolidone carboxylate, seleocysteine, selenomethionine, captopril, zofenopril, tiopronin, penicillamine, L-cysteine, gluthatione, dithiothreitol, thi orphan, cysteamine, bucillamine, dimercaprol, 1,1-ethanedi thiol, dimercaptosuccinic acid, furan-2-ylmethanethiol, omapatrilat, ovothiol A, rentiapril, thiosalicylic acid, tixocortol, mycothiol, coenzyme A, coenzyme B, disulfiram, psammaplin A, dixanthogen, pantethine, fursultiamine, octotiamine, sulbutiamine, prosultiamine, thiram, lipoic acid, lenthionine, ajoene, allicin, gemopatrilat, thioethanol, thiophospholipid, thiocholesterol, 12-niercaptododecanoic acid, 23-(9-mercaptononyl)-3,6,9,12,15,18,21-heptaoxatricosanoic Acid, and sulfanegen.

In one embodiment, the keratolytic agent within the composition of this invention and methods of use thereof is in a concentration of between 0.1% w/w to about 40% w/w of said composition.

In some embodiments, the topical composition further comprises at least one solvent. In one embodiment, the at least one solvent is selected from DMSO (dimethylsulfoxide), ethanol, isopropyl alcohol, propylene glycol, dimethyl isosorbide, isopropyl myristate, oleic acid, a polyethylene glycol, hexylene glycol, glycerin, glycofurol and combinations thereof. Each possibility represents a separate embodiment of this invention.

In some embodiments, the topical composition further comprises at least one additional active agent. In some embodiments, the topical composition further comprises at least one additional first active agent (in combination with the EGFR inhibitor; or in combination with the EGFR inhibitor and the at least second active agent) and the first active agent, or as combination with the EGFR inhibitor) selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w. Each possibility represents a separate embodiment of this invention.

In some embodiments, the topical composition further comprises at least one additional second active agent (in combination with the EGFR inhibitor; or in combination with the EGFR inhibitor and the at least first active agent) selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w. Each possibility represents a separate embodiment of this invention.

In some embodiments, the at least one additional active agent is tapinarof, in a concentration of from about 0.01% to about 1%, from about 1% to about 3%, from about 3% to about 5% w/w.

In some embodiments, the topical composition further comprises at least one ingredient selected from a moisturizer, a skin barrier, urea, ammonium lactate and combinations thereof, in a concentration of from about 0.01% w/w to about 1% w/w, from about 1% w/w to about 3% w/w or from about 3% w/w to about 5% w/w.

In some embodiments, the at least one EGFR inhibitor is selected from erlotinib, gefitinib, lapatinib, their salts, hydrates or solvates and combinations thereof. In one embodiment, the at least one EGFR inhibitor is erlotinib hydrochloride.

Exemplary dosages, strengths and concentrations of erlotinib, or erlotinib hydrochloride in the topical compositions of this invention, are in the range of from about or at 0.1% w/w, 0.5% w/w, 0.75% w/w, 1% w/w 1.5% w/w, 2% w/w, 2.5% w/w, 3% w/w, 3.5% w/w, 4% w/w, 4.5% w/w, 5% w/w, 5.5% w/w, 6% w/w 7% w/w, 8% w/w, 9% w/w, 10% w/w, 15% w/w 20% w/w or any ranges thereof of erlotinib, or erlotinib hydrochloride.

In some embodiments, the at least one PDE4 inhibitor is selected from roflumilast, apremilast, piclamilast, ibudliast, cilomilast their salts, hydrates or solvates and combinations thereof.

In some embodiments, the Janus kinase inhibitor (JAK inhibitor) is selected from tofacitinib, abrocitinib, ruxolitinib, delgocitinib, oclacitinib, baricitinib, peficitinib, or salt thereof and combinations thereof. In another embodiment, the Janus kinase inhibitor (JAK inhibitor) is tofacitinib or salt thereof. In another embodiment, the Janus kinase inhibitor (JAK inhibitor) is tofacitinib citrate.

Some of the above additional active agents, selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, play the role of avoiding, preventing or alleviating the EGFR inhibitor cutaneous side-effects.

According to some embodiments, the topical composition of this invention is a cream, an ointment, a gel, a lotion, a spray, a shampoo, a patch or a foam.

In one embodiment, the at least one EGFR inhibitor is erlotinib hydrochloride and the composition is formulated as a topical gel.

In one embodiment, the at least one EGFR inhibitor is erlotinib hydrochloride and the composition is formulated as an ointment.

In one embodiment, there is provided a topical composition for treatment, prevention or alleviation of a hyperkeratinization disorder in a patient in need thereof, comprising about 0.75% w/w erlotinib hydrochloride and from about 10% to about 98% w/w at least one penetration enhancer, or at least one keratolytic agent, or combination thereof, where the composition is formulated as a gel. In another embodiment, the composition comprises about 5% w/w erlotinib hydrochloride, about 63 wt % PEG 400, about 25 wt % PEG 3350 about 4 wt % about 0.5 wt % 2-phenoxyethanol and about 0.25 wt % methylparaben and the composition is formulated as an ointment. In another embodiment, the composition comprises between 0.1%-40% w/w keratolytic agent.

In one embodiment, there is provided a topical composition for treatment, prevention or alleviation of psoriasis in a patient in need thereof, comprising about 5% w/w erlotinib hydrochloride and from about 10% to about 98% w/w at least one penetration enhancer, or at least one keratolytic agent, or combination thereof, where the composition is formulated as an ointment. In another embodiment, the composition comprises about 5 wt % w/w erlotinib hydrochloride, about 63 wt % PEG 400, about 25 wt % PEG 3350 about 4 wt % about 0.5 wt % 2-phenoxyethanol and about 0.25 wt % methylparaben and the composition is formulated as an ointment. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tofacitinib citrate and from about 10% w/w to about 98% w/w at least one penetration enhancer, or at least one keratolytic agent, or combination thereof. In another embodiment, the composition comprises between 0.1%-40% w/w keratolytic agent.

In one embodiment, there is provided a topical composition for treatment, prevention or alleviation of a hyperkeratinization disorder in a patient in need thereof, comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5%, from 4% to about 6% w/w, w/w or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% w/w at least one penetration enhancer or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In one embodiment, there is provided a topical composition for use in the treatment, prevention or alleviation of psoriasis, wherein the topical composition comprises from about 0.5% w/w to about 3%, from about 3% w/w to about 5% w/w, from about 5% w/w to about 10% w/w, or from about 10% w/w to about 20% w/w erlotinib hydrochloride, from about 0.01% w/w to about 1% w/w roflumilast and from about 10% w/w to about 98% w/w at least one penetration enhancer, or at least one keratolytic agent, or combination thereof. In another embodiment, the composition comprises between 0.1%-40% w/w keratolytic agent.

In some embodiments, there is provided an injectable composition for treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa comprising at least one EGFR inhibitor selected from the group consisting of erlotinib, gefitinib, lapatinib, cetuximab, panitumumab, vandetanib, necitumumab, osimertinib and combinations thereof.

In another embodiment, the concentration of the EGFR inhibitor in the injectable composition is from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w.

In one embodiment, the injectable composition further comprises at least one solvent as described hereinabove.

In one embodiment, the injectable composition further comprises at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In one embodiment, the injectable composition further comprises at least one additional second active agent (in addition to the EGFR inhibitor and the first active agent, or as combination with the EGFR inhibitor) co selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w.

In some embodiments, the injectable composition further comprises (i) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and (ii) comprises at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3% or from about 3% to about 5% w/w.

In some embodiments, there is provided a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of a therapeutically effective amount of the composition as described hereinabove. In one embodiment, the composition comprises at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 5% to about 10% w/w, from 4% to about 6% w/w, or from about 10% to about 20% w/w. In one embodiment, the composition is topical.

In some embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w. In other embodiments, the method comprises daily, twice daily or three times per day topical application of therapeutically effective amounts of erlotinib or pharmaceutically acceptable salt thereof to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w, wherein said method does not induce cutaneous toxicity, or induces reduced cutaneous toxicity of erlotinib or pharmaceutically acceptable salt thereof. In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 20% w/w, wherein said method does not induce cutaneous toxicity, or induces reduced cutaneous toxicity of erlotinib or pharmaceutically acceptable salt thereof compared to erlotinib or pharmaceutically acceptable salt thereof administered systemically. In other embodiment, the psoriasis is palmoplantar psoriasis. In other embodiments, the method comprises daily, twice daily or three times per day topical application of therapeutically effective amounts of erlotinib or pharmaceutically acceptable salt thereof to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 3% w/w, from about 3% w/w to about 5% w/w, from 4% to about 6% w/w, from about 5% w/w to about 10% w/w, or from about 10% w/w to about 20% w/w erlotinib or pharmaceutically acceptable salt thereof and from about 10% to about 98% w/w at least one penetration enhancer, or at least one keratolytic agent, or combination thereof. In another embodiment, the composition comprises between 0.1%-40% w/w keratolytic agent. In other embodiments, the psoriasis is palmoplantar psoriasis. In other embodiments, the method comprises daily, twice daily or three times per day topical application of therapeutically effective amounts of the said composition to the skin portion of the subject affected by psoriasis until the skin or mucosal disorder is cured, prevented or alleviated or according to doctor's instructions.

In other embodiments, this invention provides a method of treatment, prevention or alleviation of psoriasis, by topical or injectable administration to a subject in need thereof a composition comprising erlotinib or pharmaceutically acceptable salt thereof in a concentration of from about 0.5% to about 3% w/w, from about 3% w/w to about 5% w/w, from 4% to about 6% w/w, from about 5% w/w to about 10% w/w, or from about 10% w/w to about 20% w/w erlotinib or pharmaceutically acceptable salt thereof and from about 10% to about 98% w/w at least one penetration enhancer, or at least one keratolytic agent, or combination thereof; wherein the method does not induce or induces reduced cutaneous side-effects as compared with systemic administration of the same amount of erlotinib or pharmaceutically acceptable salt thereof and wherein said method does not induce cutaneous toxicity, or induces reduced cutaneous toxicity of erlotinib or pharmaceutically acceptable salt thereof compared to erlotinib or pharmaceutically acceptable salt thereof administered systemically. In other embodiment, the psoriasis is palmoplantar psoriasis. In other embodiments, the method comprises daily, twice daily or three times per day topical application of therapeutically effective amounts of the said composition to the skin portion of the subject affected by psoriasis until the skin or mucosal disorder is cured, prevented or alleviated or according to doctor's instructions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof
(i) a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w, wherein the two separate compositions are administered concomitantly or sequentially, in either order.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof, in a concentration of 0.5% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w. In other embodiment, the psoriasis is palmoplantar psoriasis.

In other embodiment, said erlotinib and said at least one additional first active agent exhibit an additive or synergistic effect.

In some embodiments, the compositions are topical compositions.

In one additional embodiment, this invention is directed to a method of treatment, prevention or alleviation of psoriasis, by topical or injectable administration to a subject in need thereof
(i) a composition comprising a therapeutically effective amount of erlotinib or pharmaceutically acceptable salt thereof, in a concentration of 0.5% to about 20% w/w; and
(ii) a composition comprising a therapeutically effective amount of at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w. In other embodiment, the psoriasis is palmoplantar psoriasis.

In another embodiment, the method comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w, wherein the two separate compositions are administered concomitantly or sequentially, in either order.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of

a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 10%, from about 1% to about 20%, from about 0.01% to about 1%, from about 1% to about 3%, from about 3% to about 5% w/w, from about 5% to about 10% w/w, from 4% to about 6% w/w, or from about 10% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In some embodiments, the composition is a topical composition.

In another embodiment, the method further comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of
(i) a composition comprising a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w, wherein the two separate compositions are administered concomitantly or sequentially, in either order. In some embodiments, the compositions are topical compositions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of

a composition comprising:
(i) a therapeutically effective amount of at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof; in a concentration of from about 0.01% to about 1%, from about 1% to about 3%, from about 3% to about 5% w/w.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof, in a concentration of from 0.5% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis. In other embodiment, said erlotinib and said at least one additional second active agent exhibit an additive or synergistic effect.

In some embodiments, the composition is a topical composition.

In another embodiment, the method further comprises administering a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

In one additional embodiment, this invention is directed to a method of treatment, prevention or alleviation of psoriasis, by topical or injectable administration to a subject in need thereof
(i) a composition comprising a therapeutically effective amount of erlotinib or pharmaceutically acceptable salt thereof, in a concentration of from 0.5% to about 20% w/w; and
(ii) a composition comprising a therapeutically effective amount of at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis. In other embodiment, said erlotinib and said at least one additional second active agent exhibit an additive or synergistic effect.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof
(i) a composition comprising a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w;
(ii) a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof in a concentration of from about 0.01% to about 1%, from about 1% to about 3%, from about 3% to about 5% w/w;
wherein the three separate compositions are administered concomitantly or sequentially, in either order.

In one additional embodiment, this invention is directed to a method of treatment, prevention or alleviation of psoriasis, by topical or injectable administration to a subject in need thereof

a composition comprising:
(i) a therapeutically effective amount of erlotinib or pharmaceutically acceptable salt thereof in a concentration of from about 0.5% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis.

In some embodiments, the compositions are topical compositions.

In one additional embodiment, this invention is directed to a method of treatment, prevention or alleviation of a skin or mucosal disorder selected from the group consisting of psoriasis, palmoplantar psoriasis, acquired palmoplantar keratosis, eczema, ichtyosis vulgaris, non-melanoma skin cancer, actinic keratosis, a keratinization skin disorder, a keratinization mucosal disorder, pachyonychia congenita, hidradenitis suppurativa, Gorlin syndrome, prurigo nodularis and prurigo pigmentosa by topical or injectable administration to a subject in need thereof of

a composition comprising:
(i) a therapeutically effective amount of at least one EGFR inhibitor selected from the group consisting of erlotinib, gefitinib, lapatinib, cetuximab, panitumumab, vandetanib, necitumumab, osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

In some embodiments, the composition is a topical composition.

In one additional embodiment, this invention is directed to a method of treatment, prevention or alleviation of psoriasis, by topical or injectable administration to a subject in need thereof
(i) A composition comprising a therapeutically effective amount of erlotinib or pharmaceutically acceptable salt thereof in a concentration of from about 0.5% to about 20% w/w;
(ii) A composition comprising a therapeutically effective amount of at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) A composition comprising a therapeutically effective amount of at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w. In other embodiment, the psoriasis is palmoplantar psoriasis.

In one embodiment, the at least one EGFR inhibitor and at least one additional first and/or second active agent within the above methods exhibit an additive or synergistic effect.

In some embodiments, the skin or mucosal disorder treated, prevented or alleviated within the methods described hereinabove is psoriasis.

In some embodiments, the skin or mucosal disorder treated, prevented or alleviated within the methods described hereinabove is palmoplantar psoriasis.

In some embodiments, the skin or mucosal disorder treated, prevented or alleviated within the methods described hereinabove is selected from a keratinization skin disorder and a keratinization mucosal disorder. In another embodiment, the keratinization skin disorder is hyperkeratinization skin disorder.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising about 0.75% w/w erlotinib hydrochloride, about 70% w/w DMSO, about 25% w/w propylene glycol, about 0.5% w/w 2-phenoxyethanol, about 0.25% w/w methylparaben and about 3% w/w Carbopol 980, wherein the composition is formulated as a gel. In another embodiment, the method of treating, preventing or alleviating hyperkeratinization disorder comprises topical administration to a subject in need thereof of a therapeutically effective amount of a topical composition comprising about 5% w/w erlotinib hydrochloride wherein the composition is formulated as an ointment.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3% w/w, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In another embodiment, the method of treating, preventing or alleviating hyperkeratinization disorder comprises topical administration to a subject in need thereof of a therapeutically effective amount of a topical composition comprising from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.1% w/w to about 1% w/w, from about 1% w/w to about 3%, from about 3% w/w to about 5% w/w, from 4% to about 6% w/w, or from about 5% w/w to about 10% w/w erlotinib hydrochloride, from about 0.01% w/w to about 1% w/w, from about 1% w/w to about 3% w/w or from about 3% w/w to about 5% w/w apremilast and from about 10% w/w to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a therapeutically effective amount of a topical composition comprising from about from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a topical composition comprising from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof. In another embodiment, the composition comprises between 0.1%-40% w/w keratolytic agent.

In other embodiments, the present invention provide a method of treatment, prevention or alleviation of psoriasis, wherein the method comprises administering a therapeutically effective amount of a topical composition comprising from about 0.5% w/w to about 3%, from about 3% w/w to about 5% w/w, from about 5% w/w to about 10% w/w, or from about 10% w/w to about 20% w/w erlotinib hydrochloride, from about 0.01% w/w to about 1% w/w roflumilast and from about 10% w/w to about 98% w/w at least one penetration enhancer, or at least one keratolytic agent, or combination thereof. In another embodiment, the composition comprises between 0.1%-40% w/w keratolytic agent.

In some embodiments, the methods as described hereinabove do not induce or induces reduced cutaneous side-effects as compared with the same EGFR inhibitor amount administered in different methods/routes.

In some embodiments, the hyperkeratinization disorder treated, prevented or alleviated within the methods described hereinabove—is selected from palmoplantar psoriasis, hereditary palmoplantar keratoderma, acquired palmoplantar keratoderma, hydradenitis suppurativa, dermatitis, ichthyosis vulgaris, hereditary ichthyosis, acquired ichthyosis, actinic keratosis, a keratinization skin disorder and a keratinization mucosal disorder, pachyonychia congenita, hidradenitis suppurativa, Gorlin syndrome, prurigo nodularis, prurigo pigmentosa, nail psoriasis, non-melanoma skin cancer and precancerous skin, mucosal and nail lesions.

According to some embodiments, there is provided a method of treatment, prevention or alleviation of a skin disorder selected from the group consisting of psoriasis, palmoplantar psoriasis, acquired palmoplantar keratosis, eczema, ichtyosis vulgaris, non-melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3, a corticosteroid, calcipotriene, tapinarof and combinations thereof in a concentration of from about 0.01% to about 1%, from about 1% to about 3%, from about 3% to about 5% w/w, wherein the at least one EGFR inhibitor and the at least one additional active agent selected from a corticosteroid, calcipotriene, tapinarof and combinations thereof exhibit an additive or synergistic effect.

According to some embodiments, there is provided a method of treatment of a skin or mucosal disorder selected from the group consisting of psoriasis, palmoplantar psoriasis, non-acquired palmoplantar keratosis, eczema, ichtyosis vulgaris, melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from a corticosteroid, calcipotriene, tapinarof and combinations thereof in a concentration of from about 0.01% to about 1%, from about 1% to about 3%, from about 3% to about 5% w/w, wherein said at least one EGFR inhibitor and said at least one additional active agent exhibit an additive or synergistic effect.

According to some embodiments, there is provided a method of treatment of psoriasis by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising at least one additional active agent selected from a corticosteroid, calcipotriene, tapinarof and combinations thereof, in a concentration of, from about 0.01% to about 1%, from about 1% to about 3%, from about 3% to about 5% w/w, wherein said at least one EGFR inhibitor and said at least one additional active agent exhibit an additive or synergistic effect.

According to some embodiments, there is provided a method of treatment of a skin disorder selected from the group consisting of psoriasis, palmoplantar psoriasis, acquired palmoplantar keratosis, eczema, ichtyosis vulgaris, non-melanoma skin cancer, actinic keratosis, pachyonychia congenita, hidradenitis suppurativa, Gorlin syndrome, prurigo nodularis and prurigo pigmentosa by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein said at least one EGFR inhibitor and tapinarof exhibit an additive or synergistic effect.

According to some embodiments, there is provided a method of treatment of palmoplantar psoriasis by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from about 5% to about 10% w/w or from about 10% to about 20% w/w, further comprising tapinarof in a concentration of from about 0.01% to about 1%, from about 1% to about 3% or from about 3% to about 5% w/w, from 4% to about 6% w/w, wherein said at least one EGFR inhibitor and tapinarof exhibit an additive or synergistic effect.

According to some embodiments, there is provided a method any one of treatment of palmoplantar psoriasis by topical administration to a subject in need thereof a therapeutically effective amount of a composition comprising a EGFR inhibitor wherein the EGFR inhibitor is erlotinib in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein erlotinib and tapinarof exhibit an additive or synergistic effect.

According to some embodiments, there is provided a method of treatment of a skin or mucosal disorder, wherein the skin or mucosal disorder is selected from a keratinization skin disorder and a keratinization mucosal disorder by topical administration to a subject in need thereof a therapeutically effective amount of a composition comprising at least one EGFR inhibitor selected from the group consisting of erlotinib, gefitinib, lapatinib, cetuximab, panitumumab, vandetanib, necitumumab, Osimertinib or pharmaceutically acceptable salt thereof and combinations thereof, in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w, from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein said at least one EGFR inhibitor and tapinarof exhibit an additive or synergistic effect.

According to some embodiments, there is provided a method of treatment of a keratinization skin disorder or a keratinization mucosal disorder by topical administration to a subject in need thereof a therapeutically effective amount of a composition comprising a EGFR inhibitor wherein the EGFR inhibitor is erlotinib in a concentration of from about 0.01% to about 20% w/w, from about 0.01% to about 10% w/w, from about 0.1% to about 20% w/w, from about 0.1% to about 10% w/w, from about 0.5% to about 20% w/w, from about 1% to about 20% w/w from about 0.01% to about 1%, from about 1% to about 3%, from about 3% to about 5% w/w, from 4% to about 6% w/w, from about 5% to about 10% w/w or from about 10% to about 20% w/w, further comprising tapinarof in a concentration of from about 0.01% to about 1%, from about 1% to about 3% or from about 3% to about 5% w/w, wherein erlotinib and tapinarof exhibit an additive or synergistic effect.

In some embodiments, there is provided a method of treatment comprising daily, twice daily or three times per day application of therapeutically effective amounts of the composition or the two or three separate compositions to the skin portion of the subject affected by the said skin or mucosal disorder until the skin or mucosal disorder is cured, prevented or alleviated or according to doctor's instructions. In one embodiment, the method comprises daily, twice daily or three times per day topical application of therapeutically effective amounts of the topical composition or the two or three separate topical compositions to the skin portion of the subject affected by the said skin or mucosal disorder until the skin or mucosal disorder is cured, prevented or alleviated or according to doctor's instructions.

In one embodiment, the administration of the compositions within the above methods is topical or injectable administration (subcutaneous, intramuscular, intravenous, intraperitoneal, intracardiac, intraarticular, intracavernous or intralesional administration, each represents a separate embodiment) intramuscular, intravenous, intraperitoneal, intracardiac, intraarticular, intracaverno us or intralesional administration, each represents a separate embodiment). In another embodiment, the administration is topical or intralesional. In another embodiment, intralesional administration is done by regular injections or subcutaneous injections with microneedles. In another embodiment, the compositions are topical or injectable. Each possibility represents a separate embodiment of this invention. In some other embodiments, the EGFR inhibitor in any of the methods and compositions of this invention is erlotinib or salt thereof. In another embodiment the EGFR inhibitor is Erlotinib HCl. In other embodiments the erlotinib salts include a salt with an inorganic or organic acid, such as, hydrochloric, hydrobromic, sulphuric, phosphoric, methanesulfonic, benzenesulfonic, trifluoroacetic, citric, lactic, maleic acid, tosylic or oxalic acid.

As EGFR inhibitors in general and erlotinib in particular are poorly soluble, the compositions of this invention need to comprise a high EGFR inhibitor concentration of up to 20% w/w. The compositions are in the form of partly solubilized suspensions and may comprise organic solvents and solubility enhancers as well as other ingredients (e.g. penetration enhancer) and active agents which are all described hereinabove. In one embodiment, the at least one EGFR inhibitor is partly or entirely solubilized.

Unexpectedly, it was found that within the compositions of this invention, EGFR inhibitors were at least partially solubilized and used in solution/suspension form, thus the inhibitors could be used in topical or injectable compositions. Further, it was found that the compositions of this invention, when administered topically or intralesional injected within the methods described hereinabove—do not induce or induces reduced cutaneous side-effects as compared with the same EGFR inhibitor amount administered in different methods/routes.

### Definitions

As used herein, the terms “pharmaceutically active agent” or “active agent” or “active pharmaceutical ingredient” or “API” are interchangeable and mean the ingredient is a pharmaceutical drug which is biological active and is regulatory approved or approvable as such.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “10 μm” is intended to mean “about 10 μm”.

As used herein, numerical ranges preceded by the term “about” should not be considered to be limited to the recited range. Rather, numerical ranges preceded by the term “about” should be understood to include a range accepted by those skilled in the art for any given element in microcapsules or formulations according to this invention.

The term “about” as used herein means within an acceptable error range for a particular value as determined by one of ordinary skill in the art, which will depend in part on how the value is measured or determined, i.e., the limitations of the measurement system. For example, “about” can mean a range of up to 10%, more preferably up to 5%, and still more preferably up to 1% of a given value. Where particular values are described in the application and claims, unless otherwise stated, the meaning of the term “about” is within an acceptable error range for the particular value.

The terms “comprise”, “comprising”, “includes”, “including”, “having” and their conjugates mean “including but not limited to”.

The term “consisting of” means “including and limited to”.

The term “consisting essentially of” means that the composition, method or microcapsules may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

### EXAMPLES

### Example 1

### Preparation and Stability of a 0.75% Topical Erlotinib HCl Gel Composition

0.75% erlotinib; 70% DMSO;

Composition:

| Ingredient | % in formulation |
|---|---|
| Erlotinib hydrochloride | 0.75 |
| DMSO | 70 |
| Propylene glycol | 25.50 |
| 2-phenoxyethanol | 0.5 |
| Methylparaben | 0.25 |
| Carbopol 980 | 3 |

Procedure:
Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

Stability Results

| | Time | 1 month at | 2 months at | 3 months at |
|---|---|---|---|---|
| | zero | 40 C. | 40 C. | 40 C. |
| Erlotinib | 0.70% | 0.71% | 0.71% | 0.73% |
| assay | | | | |

### Example 2

### Preparation and Stability of a 0.5% Topical Erlotinib HCl Gel Composition

0.5% erlotinib; 70% DMSO;

Composition:

| Ingredient | % in formulation |
|---|---|
| Erlotinib hydrochloride | 0.5 |
| DMSO | 70 |
| Propylene glycol | 25.75 |
| 2-phenoxyethanol | 0.5 |
| Methylparaben | 0.25 |
| Carbopol 980 | 3 |

Procedure:
Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

Stability Results

| | Time zero | 2 months at 40 C. | 3 months at 40 C. |
|---|---|---|---|
| Erlotinib assay | 0.46% | 0.47% | 0.46% |

### Example 3

### Preparation and Stability of a 0.5% Topical Erlotinib HCl Gel Composition

0.5% erlotinib; 45.5% DMSO

Composition:

| Ingredient | % in formulation |
|---|---|
| Erlotinib hydrochloride | 0.5 |
| DMSO | 45.5 |
| Propylene glycol | 50.25 |
| 2-phenoxyethanol | 0.5 |
| Methylparaben | 0.25 |
| Carbopol 980 | 3 |

Procedure:
Erlotinib hydrochloride was dissolved in DMSO at 40° C.
Methylparaben was added under stirring
Carbopol was added under stirring
2-phenoxyethanol was dissolved in propylene glycol and added
The formulation was stirred and homogenized to obtain a homogeneous gel.

Stability Results

| | Time zero | 2 months at 40 C. | 3 months at 40 C. |
|---|---|---|---|
| Erlotinib assay | 0.47% | 0.47% | 0.46% |

### Example 4

### Preparation and Stability of a 0.5% Topical Erlotinib HCl Gel Composition

0.5% erlotinib; 50% EtOH 70%

Composition:

| Ingredient | % in formulation |
|---|---|
| Erlotinib hydrochloride | 0.5 |
| EtOH 70% | 50 |
| Propylene glycol | 46.25 |
| 2-phenoxyethanol | 0.5 |
| Methylparaben | 0.25 |
| Carbopol 980 | 2.5 |

Procedure:
Erlotinib hydrochloride was dissolved in EtOH at 40° C.
Methylparaben was added under stirring
Carbopol was added under stirring
2-phenoxyethanol was dissolved in propylene glycol and added
The formulation was stirred and homogenized to obtain a homogeneous gel.

Stability Results

| | Time zero | 2 weeks at 40 C. |
|---|---|---|
| Erlotinib assay | 0.47% | 0.48% |

### Example 5

### Preparation and Stability of a 1.25% Topical Erlotinib HCl Gel Composition

1.25% erlotinib; 95% DMSO;

Composition:

| Ingredient | % in formulation |
|---|---|
| Erlotinib hydrochloride | 1.25 |
| DMSO | 95 |
| 2-phenoxyethanol | 0.5 |
| Methylparaben | 0.25 |
| Carbopol 980 | 3 |

Procedure
Erlotinib hydrochloride was dissolved in DMSO at 40° C.
Methylparaben was added under stirring.
Carbopol was added under stirring.
2-phenoxyethanol was dissolved in propylene glycol and added
The formulation was stirred and homogenized to obtain a homogeneous gel.

### Example 6

### Preparation and Stability of a 1% Topical Erlotinib HCl Gel Composition

1% erlotinib; 49% PEG-400; 30% PEG-3350

Composition:

| Ingredient | % in formulation |
|---|---|
| Erlotinib hydrochloride | 1 |
| Propylene glycol | 20 |
| PEG-400 | 49 |
| PEG-3350 | 30 |

Procedure:
Propylene glycol, PEG-400 and PEG-3350 were stirred at 70% to obtain a homogeneous liquid
Erlotinib hydrochloride was added under stirring
Carbopol was added under stirring and homogenization
2-phenoxyethanol was dissolved in propylene glycol and added
The formulation was cooled to room temperature.

### Example 7

### Preparation and Stability of a 1% Erlotinib HCl+1% Tapinarof Topical Gel Composition

| Ingredient | % in formulation |
|---|---|
| Erlotinib hydrochloride | 1 |
| Tapinarof | 1 |
| DMSO | 70 |
| Propylene glycol | 24.25 |
| 2-phenoxyethanol | 0.5 |
| Methylparaben | 0.25 |
| Carbopol 980 | 3 |

Procedure:
Erlotinib hydrochloride is dissolved in DMSO at 40° C.
Tapinarof is added under stirring
Methylparaben is added under stirring
Carbopol is added under stirring
2-phenoxyethanol is dissolved in propylene glycol and added
The formulation is stirred and homogenized to obtain a homogeneous gel.

### Example 8

### Preparation of a 1% Erlotinib HCl+0.5% Tofacitinib Citrate Topical Gel Composition

| Ingredient | % in formulation |
|---|---|
| Erlotinib hydrochloride | 1 |
| tofacitinib citrate | 0.5 |
| DMSO | 70 |
| Propylene glycol | 24.75 |
| 2-phenoxyethanol | 0.5 |
| Methylparaben | 0.25 |
| Carbopol 980 | 3 |

Procedure:
Erlotinib hydrochloride is dissolved in DMSO at 40° C.
Tofacitinib citrate is added under stirring
Methylparaben is added under stirring
Carbopol is added under stirring
2-phenoxyethanol is dissolved in propylene glycol and added
The formulation is stirred and homogenized to obtain a homogeneous gel.

### Example 9

### Preparation of a 1% Erlotinib HCl+0.5% Apremilast Topical Gel Composition

| Ingredient | % in formulation |
|---|---|
| Erlotinib hydrochloride | 1 |
| Apremilast | 0.5 |
| DMSO | 70 |
| Propylene glycol | 24.75 |
| 2-phenoxyethanol | 0.5 |
| Methylparaben | 0.25 |
| Carbopol 980 | 3 |

Procedure:
Erlotinib hydrochloride is dissolved in DMSO at 40° C.
Apremilast is added under stirring
Methylparaben is added under stirring
Carbopol is added under stirring
2-phenoxyethanol is dissolved in propylene glycol and added
The formulation is stirred and homogenized to obtain a homogeneous gel.

### Example 10

### Preparation of a 5% Erlotinib HCl Ointment

| Ingredient | % in formulation |
|---|---|
| Erlotinib hydrochloride | 5 |
| PEG 400 | 62.85 |
| PEG 3350 | 25 |
| Glycerin | 4.1 |
| 2-phenoxyethanol | 0.5 |
| Methylparaben | 0.25 |
| NaOH 20% solution | 2.3 |

Procedure:
Erlotinib hydrochloride was added to PEG-400 at 50° C.
Sodium hydroxide solution was added until full dissolution is obtained.
Methylparaben, phenoxyethanol, glycerin were added under stirring.
PEG-3350 was added.
The formulation was stirred and cooled to room temperature during mixing to obtain a homogeneous ointment.

### Example 11

### Efficacy Study of Erlotinib 5% for the Treatment of Psoriasis

The objective of this study was to determine the efficacy of Erlotinib 5% as provided herein compared to the vehicle and Dexamethasone as shown at the Table below. The evaluation was based on histology analysis following application of the respective test formulations.

| | Active | Dosage |
|---|---|---|
| Group | ingredient | form |
| 1 | Vehicle | Ointment |
| 2 | Dexamethasone | Solution |
| | 2 mg | 20 |
| 3 | Erlotinib | Ointment |
| | 5% | |

The efficacy study has been conducted on female mice. The total duration of the experiment was 8 weeks, upon receiving of the human skin. Normal skin from healthy volunteers were obtained for grafting. Healthy human abdominal skin pieces with a width of 0.4 mm and surface area of 1.5×1.5 cm were provided. The skin sample was preserved in isotonic saline solution and transplanted within 6-12 hours after skin donation. In addition, blood samples were collected from psoriatic patients having classic plaque psoriasis and not undergoing any treatment. 20 mL blood samples were taken from both males and females psoriatic patients (from psoriatic patients having classic plaque psoriasis and not undergoing any treatment), and peripheral blood mononuclear cells (PBMC) were separated immediately after blood withdrawal.

Mice: Beige-severe combined immunodeficient mice C.B-17/IcrHsdscid-bg (beige-SCID) mice (weight ˜25 g) were included in this study. Mice were monitored twice a week for vital signs such as weight, food intake, coat, eyes, anal genital areas, discharge/soiling, behavior and criteria related to skin transplantation. Normal healthy human donor skin were transplanted onto the beige-SCID mice as previously described (See Keren A. et al., Novel nanosome delivery system combined with siRNA targeting the antimicrobial gene DFB4: a new approach for psoriasis management?ExpDermatol. 2014; 23:464-465; Bracke S. et al., Targeted silencing of DEFB4 in a bioengineered skin-humanized mouse model for psoriasis: development of siRNA SECosome-based novel therapies. ExpDermatol. 2014; 23:199-201; Zaretsky M. et al., Directed evolution of a soluble human IL-17A receptor for the inhibition of psoriasis plaque formation in a mouse model. Chem Biol. 2013 21; 20:202-2; Gilhar A. et al., The beneficial effect of blocking Kv1.3 in the psoriasiform SCID mouse model. J Invest Dermatol. 2011; 131:118 124; Keren A. et al., Innate lymphoid cells 3 induce psoriasis in xenotransplanted healthy human skin. J Allergy Clin Immunol. 2018; 142:305-308.e6.).

PBMC's from the psoriatic patient's blood were isolated and cultured in the presence of a high dose of IL-2; Prospec, 100 U/mL of media-RPMI 1640, 10% human AB serum (Sigma, St. Louis, Mo.), 1% glutamine, 1% antibiotics (media components; Biologicallndustries, Kibbutz Beit Haemeck, Israel), as previously described (Gilhar A. et al., The beneficial effect of blocking Kv1.3 in the psoriasiform SCID mouse model. J Invest Dermatol. 2011; 131:118-124; Nousbeck J. et al., IGFBP7 as a potential therapeutic target in Psoriasis. J Invest Dermatol. 2011; 131:1767-1770; Schafer P H. et al., Apremilast, a cAMP phosphodiesterase-4 inhibitor, demonstrates anti-inflammatory activity in vitro and in a model of psoriasis. British Journal of Pharmacology 2010; 159:842-855).

Four weeks following the engraftment, each mouse was injected (intradermally) with 1×10⁷ IL-2 enriched allogeneic PBMC's from psoriatic patients (1×10⁷ cells injected/mouse). Cells from different psoriasis patients were equally distributed between treatment groups. Each patient was represented in each treatment group.

Fourteen days following cells injections, the mice were divided into treatment groups (Table, see below) and treated according to the timeline indicated below (see Scheme 1). Each transplant was equally topically treated (50 μg) by using a sterile spatula.

Dexamethasone (D2915, Sigma): was freshly prepared for each administration by dilution in saline to final concentration (50 mg/ml). Dexamethasone serves as a positive control and was administered topically twice daily for 14 days following cells injections (at Day 42 following human skin transplantation) until the end of the study (Day 56).

The vehicle was administered topically (50 μg) once daily for 14 days as well.

On Day 56, the entire skin graft was excised and placed in 10% formaldehyde in saline overnight. Then, the specimens were placed in 70% ethanol and embedded and stained according to the standard Hematoxylin/Eosin protocol.

Skin graft histological assessment was performed (according to the psoriatic criteria including epidermal thickness) using light microscopy, and two observers blindly performed the evaluations. Epidermal thickness was determined using an ocular micrometer at a minimum of 50 points along the epidermis selected to represent points of maximal and minimal thickness. Thickness of the suprapapillary plate was measured similarly at 50 points for each sample. Three slices were examined for each sample.

### Study Results

### Histological Evaluation of Psoriasis-Induced Xenotransplants Study:

| | | | | Psoriatic | Partial | Complete |
|---|---|---|---|---|---|---|
| Group | Compound | Route | Frequency | Features | Recovery | Recovery |
| 1 | Vehicle | Topical | Once | 9/10 | 1/10 | 0/10 |
| | ointment | | daily | (90%) | (10%) | (0%) |
| 2 | Dexameth- | Topical | Twice | 0/10 | 1/10 | 9/10 |
| | asone | | a day | (0%) | (10%) | (90%) |
| | 2 mg | | | | | |
| 3 | Erlotinib | Topical | Once | 4/10 | 1/10 | 5/10 |
| | ointment | | daily | (40%) | (10%) | (50%) |
| | 5% | | | | | |

What is claimed is:

1. A method of treatment, prevention or alleviation of psoriasis, by topical or injectable administration to a subject in need thereof erlotinib or pharmaceutically acceptable salt thereof in a concentration of from about 0.5% to about 20% w/w.

2. The method of claim 1, wherein said method does not induce cutaneous toxicity, or induces reduced cutaneous toxicity of erlotinib or pharmaceutically acceptable salt thereof.

3. A method of treatment, prevention or alleviation of psoriasis, by topical or injectable administration to a subject in need thereof
a composition comprising:
(i) a therapeutically effective amount of erlotinib or pharmaceutically acceptable salt thereof, in a concentration of 0.5% to about 20% w/w; and
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

4. The method of claim 3, wherein the method further comprises administering a composition comprising at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w, wherein the two separate compositions are administered concomitantly or sequentially, in either order.

5. A method of treatment, prevention or alleviation of psoriasis, by topical or injectable administration to a subject in need thereof
a composition comprising:
(i) a therapeutically effective amount of erlotinib or pharmaceutically acceptable salt thereof, in a concentration of from 0.5% to about 20% w/w; and
(ii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

6. The method of claim 5, wherein the method further comprises administering a composition comprising at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w.

7. A method of treatment, prevention or alleviation of psoriasis, by topical or injectable administration to a subject in need thereof
a composition comprising:
(i) a therapeutically effective amount of erlotinib or pharmaceutically acceptable salt thereof in a concentration of from about 0.5% to about 20% w/w;
(ii) at least one additional first active agent selected from a corticosteroid, calcipotriene, tapinarof, a Janus kinase inhibitor (JAK inhibitor), a phosphodiesterase-4 inhibitor (PDE4 inhibitor), and combinations thereof, in a concentration of from about 0.01% to about 1%, from about 1% to about 3%, from about 3% to about 5% w/w; and
(iii) at least one additional second active agent selected from menadione, ketoconazole, dapsone, cevimeline, spironolactone, tretinoin, pimecrolimus, a tetracycline, a sunscreen, doxycycline, epidermal growth factor (EGF), lycopene, threolone, synthomycine, erythromycin, Vitamin K3 and combinations thereof, in a concentration of from about 0.01% to about 1%, from about 1% to about 3%, from about 3% to about 5% w/w.

8. The method according to claim 1, wherein the psoriasis is palmoplantar psoriasis.

9. The method according to claim 3, wherein the psoriasis is palmoplantar psoriasis.

10. The method according to claim 5, wherein the psoriasis is palmoplantar psoriasis.

11. The method according to claim 7, wherein the psoriasis is palmoplantar psoriasis.

12. The method according to claim 1, wherein the method comprises topical administration to a subject in need thereof of a therapeutically effective amount of a topical composition comprising from about 0.5% w/w to about 3% w/w, from about 3% w/w to about 5% w/w, from 4% to about 6% w/w, from about 5% w/w to about 10% w/w, or from about 10% w/w to about 20% w/w erlotinib or pharmaceutically acceptable salt thereof and from about 10% to about 98% w/w at least one penetration enhancer or at least one a keratolytic agent, or combination thereof.

13. The method according to claim 12, wherein the method does not induce or induces reduced cutaneous side-effects as compared with systemic administration of the same amount of erlotinib or pharmaceutically acceptable salt thereof and wherein said method does not induce cutaneous toxicity or induces reduced cutaneous toxicity of erlotinib or pharmaceutically acceptable salt thereof.

14. The method according to claim 1, wherein the method comprises once daily, twice daily or three times per day topical application of therapeutically effective amounts of the said composition to the skin portion of the subject affected by psoriasis until the skin or mucosal disorder is cured, prevented or alleviated or according to doctor's instructions.

15. The method according to claim 7, wherein said erlotinib and said at least one additional first and/or second active agent exhibit an additive or synergistic effect.

16. The method according to 3, wherein said erlotinib and said at least one additional first active agent exhibits an additive or synergistic effect.

17. The method according to claim 5, wherein said erlotinib and said at least one additional second active agent exhibits an additive or synergistic effect.

18. The method according to claim 7, wherein said erlotinib and said at least one additional first active agent and said at least one second active agent exhibit an additive or synergistic effect.

19. The method according to claim 3, wherein the method comprises topical administration to a subject in need thereof of a therapeutically effective amount of a topical composition comprising from about from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w, from about 3% w/w to about 5% w/w tapinarof and from about 10% to about 98% w/w at least one penetration enhancer, or at least one keratolytic agent, or combination thereof.

20. The method according to claim 3, wherein the method comprises topical administration to a subject in need thereof of a therapeutically effective amount of a topical composition comprising from about 0.5% w/w to about 3% w/w, from about 3% w/w to about 5% w/w, from about 5% w/w to about 10% w/w, or from about 10% w/w to about 20% w/w erlotinib hydrochloride, from about 0.01% w/w to about 1% w/w, from about 1% w/w to about 3% w/w or from about 3% w/w to about 5% w/w tofacitinib citrate and from about 10% to about 98% w/w at least one penetration enhancer, or at least one keratolytic agent, or combination thereof.

21. The method according to claim 3, wherein the method comprises topical administration to a subject in need thereof of a therapeutically effective amount of a topical composition comprising from about 0.5% w/w to about 3%, from about 3% w/w to about 5% w/w, from about 5% w/w to about 10% w/w, or from about 10% w/w to about 20% w/w erlotinib hydrochloride, from about 0.01% w/w to about 1% w/w roflumilast and from about 10% w/w to about 98% w/w at least one penetration enhancer, or at least one keratolytic agent, or combination thereof.
